(19) United States
(12) Patent Application Publication
(10) Pub. No.: US 2026/0079055 A1
(43) Pub. Date: Mar. 19, 2026

(54) FLAT PRESSURE SENSOR UNIT

(71) Applicant: KARMA Technologies AG, St. Gallen (CH)

(72) Inventor: Heini SEGER, Niederteufen (CH)

(73) Assignee: KARMA Technologies AG, St. Gallen (CH)

(21) Appl. No.: 19/110,521

(22) PCT Filed: Sep. 6, 2023
(86) PCT No.: PCT/EP2023/074356

§ 371 Date: Mar. 11, 2025

(30) Foreign Application Priority Data
Sep. 12, 2022 (EP) 22195124.7

(51) Int. Cl. G01L 1/14; A61B 5/00

(52) U.S. Cl. CPC G01L 1/146; A61B 5/6892

(57) ABSTRACT

The invention relates to a flat pressure sensor unit, in particular a pressure sensor sheet (3), comprising first conductor paths (310) and second conductor paths (32) and a central layer (33) arranged between the first and the second conductor paths (310, 320). The first and second conductor paths (310, 320) cross one another, spaced apart by the central layer (33), whereby cross-over points (30) are formed in order to detect, as sensors, a spacing change between the first and second conductor paths (310, 320) generated by an external application of pressure. The conductor paths (310, 320) are sewn onto at least one material layer (31, 32). First and second connecting paths (390, 391) are provided, leading away from the first and second conductor paths (310, 320), extending towards at least one electronics unit (380, 381) and ending at the at least one electronics unit (380, 381). The first and second conductor paths (390, 391) are sewn on the at least one material layer (31, 32). The flat pressure sensor unit can be easily and economically produced and allows for flexible design both in the selection of the material layers (31, 32) and in the arrangement of the conducting and connecting paths (310, 320, 390, 391).

### TECHNICAL FIELD

The present invention relates to a flat pressure sensor unit, in particular a pressure sensor sheet, in particular a textile pressure sensor sheet, and to a method for producing a pressure sensor unit of this kind.

### PRIOR ART

Optimum patient monitoring is an increasing problem in particular due to understaffing in the care sector. Technical solutions can provide a remedy here and ensure increased safety and easing of burden in patient care.

Solutions for improving sleeping comfort are also sought in the private sector.

U.S. Pat. No. 5,144,284 discloses a mat which has pressure sensors and can be laid on a bed in order to monitor the movement of a patient. WO 2012/153263 A1 relates to a sensor pillow having pressure sensors for monitoring sleep.

WO 01/75924 A1 describes pressure sensors in the form of an electrically conductive fabric. It is mentioned that a sensor fabric of this kind can be used in combination with a mattress. EP 1 269 502 B1 discloses a sensor mat having an electrically conductive layer.

EP 3 736 364 A1 discloses a textile sensor mat for a bed, wherein the sensor mat is used to determine the pressure distribution for bedridden people.

EP 3 447 463 B1 from Sefar AG discloses a textile pressure sensor. In an embodiment according to FIGS. 7 and 8, a first fabric layer has electrically conductive threads which extend in a first direction. A second fabric layer having electrically conductive threads extends in a second direction, which runs perpendicular to the first direction. A piezoresistive third fabric layer is arranged between the two fabric layers. This third fabric layer changes its resistance and thus its electrical conductivity depending on the application of pressure force. If a measurement current is applied to the electrically conductive threads and the mat is subjected to a pressure, the electrical resistance changes in the intersection regions. This change is used as a measure of the applied pressure and can be recorded by a control unit.

J. Cheng, et al., Smart-surface: Large scale textile pressure sensors arrays for activity recognition, Pervasive and Mobile Computing (2016), http://dx.doi.org/10.1016/j.pmcj.2016.01.007, discloses a sensor mat from Sefar AG having a third fabric layer, which consists of a pressure-sensitive material having the trademark name CARBOTEX® from Sefar AG. The electrically conductive threads form a grid or a matrix n×m with intersection regions. Each intersection region acts as a pressure sensor and corresponds to one pixel of the pressure distribution matrix. An FPGA (Field Programmable Gate Array) controls ultra-fast switch units (switch arrays) and analog-to-digital converters (ADC) and collects data of the matrix. Each matrix column i of n is switched on individually, while the others are deactivated. The voltages in the m rows then correspond to those at the intersection regions of the m rows of the i-th column. These voltages of the m rows are passed on to multiplexers and fed into the ADCs. In the next step, the next column i+1 is switched on and the corresponding voltages in the m rows are passed on. The way in which the individual multiplexers are controlled is not described. The column by column query results in a data series which corresponds to the pressure distribution. n ADCs and n associated multiplexers are required. It is specified that 128×128 sensor points can be passed on to a computer at 40 images per second. An image analysis is then carried out.

The as yet unpublished patent application from the same applicant, EP 21 200 161.4 of Sep. 30, 2021, describes a cost-effective and quick method and also a corresponding pressure sensor unit for querying sensor values of a pressure sensor sheet.

However, production of the above-described sensor sheets, in particular the textile sensor sheets, is also relatively time-consuming and cost-intensive.

WO 2022/146612 A1 discloses a textile material which has capacitive, inductive and NFC sensors. The sensor lines are sewn on. The material can be used as a pants pocket.

### SUMMARY OF THE INVENTION

It is therefore an object of the invention to provide a flat pressure sensor unit, in particular a sensor sheet, and a method for producing a flat pressure sensor unit which simplify production.

This object is achieved by a flat pressure sensor unit having the features of claim 1 and a method having the features as claimed in claim 14.

The pressure sensor unit according to the invention has first conductor tracks and second conductor tracks and a middle layer arranged between the first and the second conductor tracks. The first and second conductor tracks intersect in a manner spaced apart by the middle layer, as a result of which intersection points are formed in order, as sensors, to detect a change in spacing between the first and second conductor tracks, the change in spacing being generated by external application of pressure. The first and second conductor tracks are sewn onto at least one material ply. First and second connecting tracks are provided, which, in a manner leading away from the first and second conductor tracks, extend to at least one electronics unit and which end at the at least one electronics unit. The first and second connecting tracks are sewn on the at least one material ply.

In some embodiments the middle layer extends as a flat material along a plurality of first and second conductor tracks. In other forms, the middle layer is formed by individual pieces which are located with a spacing from each other. The pieces are located at least in the intersection regions of the first and second conductor tracks. Their shape is, for example, oval, round or rectangular. The total area of all spacings between the pieces is preferably several times greater than the total area of all the pieces.

The design in pieces has the advantage that the pressure sensor unit, in particular the sensor sheet, can be more flexible. It is therefore more comfortable in use. In addition, it can be folded more easily and requires less storage space.

The pressure sensor unit according to the invention is suitable for use as a pressure sensor sheet. However, it can also be connected to other articles or integrated into them. The pressure sensor unit is suitable, in particular, for use in, on or for articles for the human body to lie or sit on. For example, it may be part of a mattress, a bedframe, a lounger, an armchair, a chair, a sofa or a car seat. The terms “pressure sensor sheet” and “sensor sheet” are used in the following. However, they also include the mentioned embodiments integrated in other articles, placed onto other articles or connected to other articles.

In this text, the term “intersection point” also includes the term “intersection region”. According to this text, “sewing”, “sewing to” and “sewing on” mean securing elements, for example cables or wires, to a material ply by means of stitching. “Embroidering” in this text is the creation of patterns on the material ply by way of elements being secured to the material ply by means of stitching. The “middle layer” is also referred to as a sensor layer since it does not necessarily have to be arranged between two plies, but rather may itself form an upper layer. “Middle” is to be understood with respect to the arrangement between the first and second conductor tracks which intersect in a spaced apart manner. The term “bedframe” includes all substrates on which a mattress or a lying surface is supported.

The sensor formed at the intersection points is preferably a capacitive sensor, as described in the publications and patent applications mentioned at the outset, in particular in as yet unpublished patent EP 21 200 161.4 of Sep. 30, 2021. The intersection points or intersection regions act like capacitors or electrical resistors. The capacitance of a capacitor changes indirectly proportionally to the spacing between its two capacitor plates. Therefore, the capacitance at an intersection region changes when a person is lying on this intersection region and therefore compresses the middle layer. If an electrical voltage is applied to the first and/or second conductor tracks, the change in capacitance results in a change in resistance. This change in resistance can be detected by means of a voltage divider.

Since the first and second conductor tracks are sewn onto the at least one material ply, production is hugely simplified. It is also advantageous that the first and second conductor tracks can be arranged in any desired patterns. They form an embroidery pattern. They are therefore embroidered on.

Each conductor track is preferably embroidered on separately from the other conductor tracks. However, a connecting track is preferably also embroidered on separately from the other connecting tracks. In this text, “separately” means with separate stitches.

Since the first and second connecting tracks are likewise sewn or embroidered onto the at least one material ply, the connection to the at least one electronics unit is simplified. In particular, it is no longer necessary to arrange flexprints over an entire length or width of a sheet in order to allow cable connections that are as short as possible between the first and second conductor tracks and the electronics unit. Furthermore, it is no longer necessary to solder cables to the large number of conductor tracks. The electronics unit can then be relatively small. In particular, it can be arranged in a relatively small region of the sheet, for example in a corner region or a subregion of an edge of the sheet. The flexprints no longer have to extend over approximately the entire length and width of the sheet.

A further advantage is that the pressure sensor sheet can be folded together more effectively since the at least one electronics unit arranged on the or in the sheet is of relatively small design.

Furthermore, it is advantageous that there is a greater flexibility in the choice of material for the material ply since the first and second conductor tracks no longer have to be incorporated in the fabric.

The first and second conductor tracks and the first and second connecting tracks are preferably sewn on by means of a thread, for example a textile or plastic thread. The thread is preferably electrically non-conductive. Depending on the embodiment, the stitches are separated from each other, and therefore the thread is linked to itself after each stitch. In preferred embodiments however, a thread extending over several stitches, preferably over an entire length of a conductor track or connecting track is used. This allows a sewing or embroidery machine to be used. Depending on the embodiment, the stitches pass through the material plies or the thread runs only in an outer portion of the layer.

The use of an embroidery machine has the advantage that the pattern to be achieved for the conductor tracks and connecting tracks can be programmed into the control module of the embroidery machine in a simple manner and the pattern can be created fully automatically.

The first and second conductor tracks and also the first and second connecting tracks are preferably secured to the at least one material ply solely by being sewn onto the at least one material ply. This simplifies production.

In a simple embodiment, the first and second connecting tracks and the first and, respectively, second conductor tracks are separate components, which are connected to each other. They are soldered together or connected by crimping, for example.

In preferred embodiments however, extensions of the first and/or second conductor tracks form the first and, respectively, second connecting tracks, as a result of which the first and/or second connecting tracks are formed in one piece with the first and, respectively, the second conductor tracks. Therefore, there are no additional tracks, but rather the first and/or the second conductor tracks run as far as the at least one electronics unit. This simplifies production and reduces interference signals.

In preferred embodiments, the first conductor tracks form rows of a grid, wherein the rows have ends define two mutually opposite first faces of the grid. The second conductor tracks form columns of this grid, wherein the columns have ends which define two mutually opposite second faces of the grid. The first connecting tracks, which lead away from the first conductor tracks, lead along at least one of the two first faces of the grid to at least one of the two second faces of the grid. In the process, the first and second connecting tracks are connected to the at least one electronics unit on the at least one second face of the grid. Since the first and second connecting tracks are guided to the electronics unit outside the shape formed by the grid, they do not affect the intersection points and therefore the individual sensors. In addition, this pattern is simple and quick to sew or embroider, and therefore production is simplified once again.

In preferred embodiments, the first and second conductor tracks and/or the first and second connecting tracks are electrically conductive threads, wires or cables. They are preferably flat or round in cross section. They are preferably silver-plated copper wires or other wires which have an optimum conductivity.

The first and second conductor tracks preferably do not have any electrical insulation. Depending on the embodiment, the first and second connecting tracks are electrically insulated or likewise do not have any electrical insulation.

If the first and second conductor tracks form the first and, respectively, second connecting tracks, they are preferably electrically insulated in the region in which they form the first and second connecting tracks and they do not have any electrical insulation in the other region. In other embodiments, they do not have any electrical insulation over their entire length. This is the case particularly when the region that forms the connecting tracks runs outside the pattern that is formed by the intersection points.

The pressure sensor sheet preferably has at least one flexprint, which forms the electronics unit. Flexprints are thin, flexible printed circuit boards. They are usually etched, copper-plated films, preferably composed of plastic. The flexprints can be arranged on the sensor sheet or incorporated into the sensor sheet in a simple manner. They do not increase the thickness of the sheet or do so only to a minimal extent.

The first and second connecting tracks are connected to the at least one electronics unit by crimping. This is a simple, quick and reliable coupling of electronics components.

The material or the fabric between the conductor tracks of the individual columns is preferably electrically non-conductive, and therefore these conductor tracks are DC-isolated from each other. The same applies to the material or the fabric between the conductor tracks of the individual rows. The middle layer is therefore preferably a dielectric or electrically insulating. The middle layer is preferably homogenous. It preferably consists of a material of which the thickness in the region of the expected externally applied pressure changes proportionally and which therefore changes its electrical conductivity proportionally to the exerted pressure. The middle layer preferably consists of Carbotex®, a material sold by the company Sefar AG, or from SEFAR® PresSense, a material produced by the same company. Other materials that serve as sensor elements can likewise be used.

Depending on the embodiment, the pressure sensor sheet has different plies. In some embodiments, the first conductor tracks and the first connecting tracks are sewn on a first material ply and the second conductor tracks and the second connecting tracks are sewn on a second material ply, wherein the middle layer is arranged between the first and the second material ply. In some variants, the first material ply, the second material ply and the middle layer are jointly formed from a composite material, wherein the first and second conductor tracks and the first and second connecting tracks are sewn onto the composite material. In other variants, the first material ply, the second material ply and the middle layer are self-supporting material webs, which are joined after the first and second conductor tracks and the first and second connecting tracks are sewn on.

In a preferred embodiment, the first conductor tracks are sewn onto a self-supporting material ply. The second conductor tracks are sewn on the middle layer. The self-supporting material ply and the middle ply are preferably connected to each other by sewing on the second conductor tracks. In so doing, it is preferably important in the production process that stitches that would damage the first conductor tracks are skipped, i.e. not made. In this example, the first connecting lines and the second connecting lines are preferably sewn onto the self-supporting material ply. The electronics units, in particular the flexprints, are preferably also sewn onto the self-supporting material ply. In addition or as an alternative, the electronics units can also be adhesively bonded or secured in some other way.

In this example, the middle layer is preferably smaller than the self-supporting material ply. In this example, the self-supporting material ply is preferably a nonwoven material, a felt, a milled material or a warp-knitted fabric.

The first and second material ply are preferably a respective or a common textile material ply, preferably a woven fabric, a warp-knitted fabric, a milled material, a knitted fabric, a mesh, a knitted mat, a nonwoven material or a felt.

In the method according to the invention for producing a pressure sensor sheet according to the invention, the first and second conductor tracks are sewn onto at least one material ply in a first specified pattern and the first and second connecting tracks are sewn onto the at least one material ply in a second specified pattern, wherein they lead from the first and second conductor tracks to the at least one electronics unit and wherein the first and second connecting tracks are connected to the at least one electronics unit.

The pressure sensor sheet according to the invention is preferably, but not necessarily, arranged in a protective envelope, which surrounds the at least one material ply. The pressure sensor sheet can be arranged on a mattress, wherein it can form the uppermost ply of the bed or it is arranged between the sheet and the mattress. It can have means for fixing on a mattress, such as for example straps which are placed around the corners of the mattress, or it can be stretched over the mattress like what is known as a fitted sheet. In these or other embodiments, it is placed on the mattress and fixed by a sheet lying over it.

The pressure sensor sheet can also be placed between the mattress and the bedframe. Depending on the embodiment, it can preferably be secured to the mattress and/or to the bedframe. It can also be arranged inside the mattress or already be fixedly connected to the mattress by the manufacturer. It can also be fixedly connected to the bedframe at the manufacturing stage.

The process according to the invention of sewing on the conductor tracks and the connecting tracks or embroidering the pattern is particularly advantageous if the sensor sheet is intended to form a unit with the mattress or the bedframe. The pattern can be adapted to the corresponding loading pattern of the mattress or the bedframe in a simple manner during manufacture. That is to say, points which require special attention for a selected specific mattress or bedframe in order to allow a person to lie in as pain-free or comfortable manner as possible can be provided with corresponding sensor patterns in the sensor sheet. Sensor sheets of which the intersection points, that is to say the pressure pattern of which, are adapted to the arrangement of the springs of a spring-core mattress, the arrangement of the slats of a slatted frame, the arrangement of a mattress base of a slatted frame and the distribution of the air chambers of a foam mattress can be produced in a simple manner. The number of intersection points that a sensor sheet is intended to have can also be chosen in a flexible manner. If the pressure sensor unit is intended to be used in one of the other, abovementioned fields of application, the intersection points can be arranged in accordance with the requirements of this field of application during production of the pressure sensor unit.

Further embodiments are laid down in the dependent claims.

### BRIEF DESCRIPTION OF THE DRAWINGS

Preferred embodiments of the invention are described in the following on the basis of the drawings, which serve solely for explanation and are not to be interpreted as restrictive. In the drawings:

FIG. 1 shows a schematic representation of a patient bed with a pressure sensor sheet according to the invention of a pressure sensor system according to the invention;

FIG. 2 shows a schematic representation of the pressure sensor sheet according to the invention in a first embodiment;

FIG. 3 shows a schematic representation of the pressure sensor sheet according to the invention in a second embodiment;

FIG. 4 shows a schematic representation of the pressure sensor sheet according to the invention with connecting tracks and flexprints;

FIG. 5 shows a photograph of a portion of the sheet according to the invention in a third embodiment;

FIG. 6 shows a photograph of a portion of the sheet according to FIG. 5;

FIG. 7 shows a schematic representation of a bed structure having a pressure sensor sheet according to the invention in a first variant of use;

FIG. 8 shows a schematic representation of a bed structure having a pressure sensor sheet according to the invention in a second variant of use;

FIG. 9 shows a schematic representation of a bed structure having a pressure sensor sheet according to the invention in a third variant of use;

FIG. 10 shows a schematic representation of a bed structure having a pressure sensor sheet according to the invention in a fourth variant of use;

FIG. 11 shows a schematic representation of a bed structure having a pressure sensor sheet according to the invention in a fifth variant of use;

FIG. 12 shows a schematic representation of a bedframe according to the prior art; and

FIG. 13 shows a schematic representation of a use of a pressure sensor sheet according to the invention on the bedframe according to FIG. 11.

### DESCRIPTION OF PREFERRED EMBODIMENTS

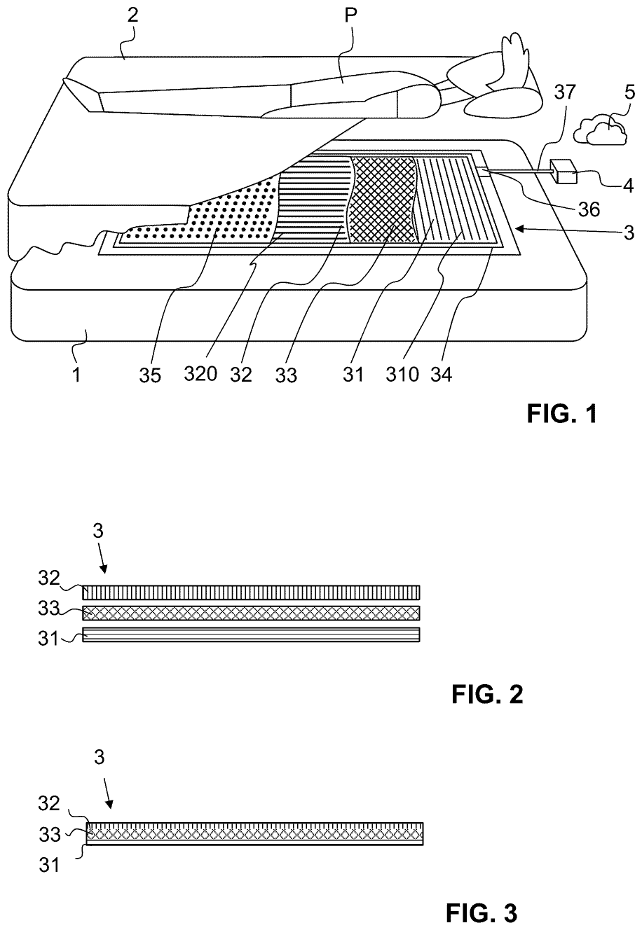

FIG. 1 schematically shows a bed, in particular a patient bed, with a pressure sensor sheet according to the invention.

A mattress 1 is covered, as usual, with a bedsheet 2, on which a patient P is lying. A pressure sensor sheet 3 according to the invention is placed between the mattress 1 and the bedsheet 2. The pressure sensor sheet 3 may be, for example, in the form of a fitted sheet or, as shown here, in the form of a flat flexible and preferably soft mat. Depending on the embodiment, it extends over the entire surface of the mattress 1 or ends, as shown here, spaced apart from the mattress edge. In other embodiments, the pressure sensor sheet 3 only extends in a subregion of the mattress 1, for example only in the upper or in the middle or in the lower region of the mattress surface. In further embodiments, the pressure sensor sheet 3 is part of the mattress 1 or the bedsheet 2 or it is part of a bedframe or is supported on it. The arrangement of the sensor sheet beneath the mattress, for example on a support for the mattress or on a bedframe is advantageous since changing the bedding, in particular the bedsheet 2, and also the lying properties of the mattress and the lying comfort of the patient are not affected. The same applies when the pressure sensor unit according to the invention is used in other articles and objects, for example the sitting comfort and the sitting properties in an armchair or on a chair are not affected given a corresponding arrangement.

In this example, the pressure sensor sheet 3 is connected to a control device 4 via a connection 36 and a cable 37, preferably a 6-pole cable. A control unit of the control device is connected to a DC voltage source. Furthermore, it is connected to a data memory unit and/or a data evaluation unit. These can be arranged in one unit in situ and/or can be cloud-based. A cloud is provided with reference sign 5 in FIG. 1. The control device 4 is preferably implemented as an SPI master, preferably on a Raspberry Pi.

As can be clearly seen in FIG. 1, the pressure sensor sheet 3 has a first material ply 31, in particular a fabric ply, a second material ply 32, preferably likewise a fabric ply, and a middle layer 33 arranged therebetween. The two material plies 31, 32 and the middle layer 33 are preferably connected, preferably adhesively bonded, to each other at points. They are preferably jointly surrounded by a protective envelope, wherein the envelope preferably has a lower ply 34 and an upper ply 35, which consist of the same material or of different materials, depending on the embodiment. They are preferably soft and flexible, in particular a textile fabric or another suitable material ply. Depending on the field of application, at least the upper of the two plies 34, 35 is preferably provided with incontinence protection, so that the three intermediate plies 31, 32, 33 are protected from moisture. In other embodiments, there is no protective envelope, for example when the pressure sensor sheet is incorporated in a mattress.

The first and the second material ply 31, 32 are preferably manufactured from the same material. They are preferably manufactured from an electrically non-conductive material. They are preferably a woven fabric, a warp-knitted fabric, a milled material, a knitted fabric, a mesh, a knitted mat, a nonwoven material or a felt.

The middle layer 33 is manufactured from a pressure-sensitive, preferably dielectric, material. The middle layer is preferably homogenous. It preferably consists of a material of which the thickness in the region of the expected externally applied pressure changes proportionally and which therefore changes its electrical conductivity proportionally to the exerted pressure.

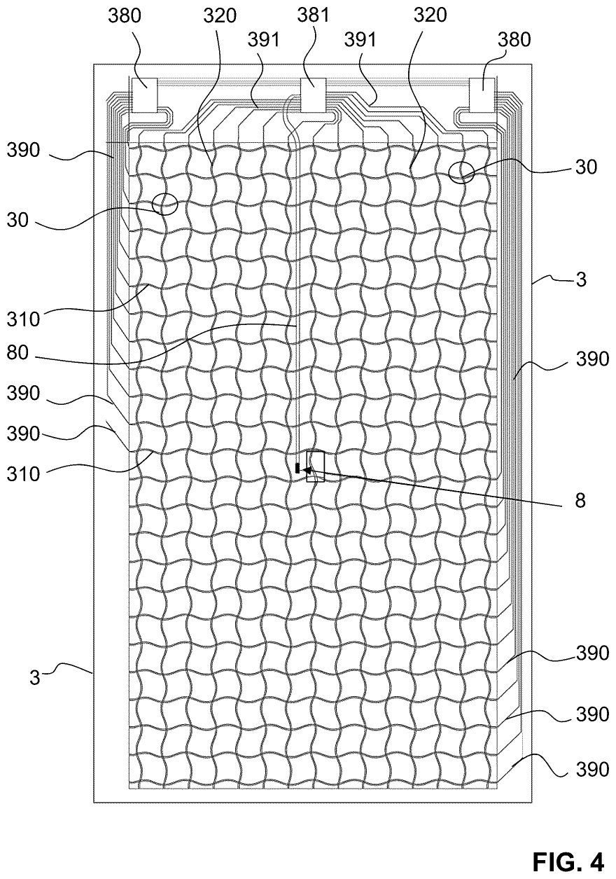

Electrically conductive first conductor tracks 310 are arranged on the first material ply 31 and electrically conductive second conductor tracks 320 are arranged on the second material ply 32. They are sewn on or to the respective material ply 31, 32. The individual first conductor tracks 310 preferably run parallel to each other and the individual second conductor tracks 320 likewise run preferably parallel to each other. However, the first conductor tracks 310 preferably run perpendicularly to the second conductor tracks 320. They thus form a grid or a matrix. This can be clearly seen in FIG. 4.

In FIG. 1, the first and second conductor tracks 310, 320 are rectilinear. In FIG. 4, they run with a waved shape. Both variants and other profiles are possible.

Furthermore, electrically conductive first connecting tracks 390 are provided on the first material ply 31 and electrically conductive second connecting tracks 391 are provided on the second material ply 32, these likewise being sewn onto the respective material ply 31, 32. These first and second connecting tracks 390, 391 are not shown in FIG. 1. However, they can be seen in FIG. 4. The first and second connecting tracks 390, 391 are preferably one-piece extensions of the first and, respectively, second conductor tracks 310, 320. The first connecting tracks 390 preferably run along one, or as in this example, on two opposite first faces of the grid, which is formed by the first and second conductor tracks 310, 320, to a second face of the grid. The second connecting tracks 291 are located on this face. The first and second connecting tracks 290, 291 are connected to at least one electronics unit on this face. This is preferably a flexprint. In this example, two first flexprints 380 are provided for connection to the first connecting tracks 390 and a second flexprint 381 is provided for connection to the second connecting tracks 391. The flexprints 380, 381 are arranged either on the associated material ply 31, 32 or between the two material plies 31, 32.

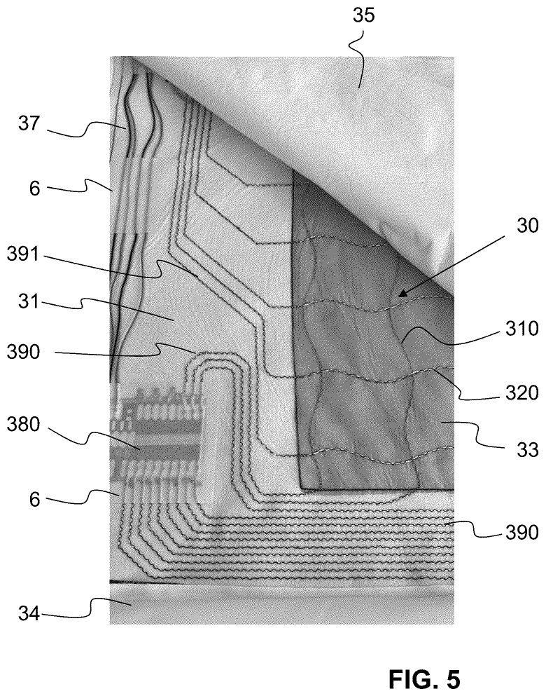

The way in which the first and second connecting tracks 390, 391 are sewn onto the first material ply 31 can be seen in FIG. 5. Each first and second connecting track 390, 391 is fixed, that is to say sewn, onto the material ply 31 with a dedicated thread 392 with several stitches. The first and second conductor tracks 310, 320 can be sewn on in the same way. One thread 392 in each case is preferably used over the entire length of the corresponding track. Sewing is preferably performed by machine. A sewing machine of a known type can be used for this purpose. However, an embroidery machine is preferably used.

As can be seen in FIG. 4, the first and second conductor tracks 310, 320 have different orientations. The first conductor tracks 310 run perpendicularly to the second conductor tracks 320. Intersection regions or intersection points 30 are thus produced. In FIG. 4, two points are encircled so that they can be seen more clearly. The conductor tracks 310, 320 do not contact each other in the intersection points 30 since they are separated from each other by the middle layer 33. The intersection points 30 thus act as capacitors or resistors, as explained in the introductory part. The capacitance of a capacitor changes indirectly proportionally to the spacing between its two capacitor plates. The capacitance at an intersection point 30 thus changes when a person rests on this intersection point 30 and thus compresses the middle layer 33.

The change in capacitance results in a change of the resistance, which is detected by means of a voltage divider in this example. For this purpose, a constant DC voltage is preferably applied to the first or to the second conductor tracks 310, 320. The voltage is preferably 3.3 V and thus does not affect the wellbeing of the patient. Other voltages can likewise be used.

In order to determine the lying position of the patient, each intersection region 30, that is to say each sensor, is individually queried. For this purpose, the electrical voltage is applied only to one row, that is to say to a first conductor track 310, in each case, the values of all columns, that is to say the second conductor tracks 320, are read out in succession and only then is the next row “energized”, that is to say has electrical voltage applied to it. The other rows or first conductor tracks 310 are set to 0 V. Accordingly, all rows are cycled through in succession. However, other types of sensor read out are possible.

Further sensors are provided in some embodiments. In FIG. 4, a temperature sensor 8 is provided in the sensor sheet 3. The sensor lines from the temperature sensor 8 to the electronics unit, here to the second flexprint 381, are provided with reference sign 80.

In the described example, the first material ply 31, the second material ply 32 and the middle layer 33 are each separate elements, which are individually self-supporting. This is shown in FIG. 2. The conductor tracks 310, 320 and connecting tracks 390, 391 or the combined and one-piece conductor/connecting tracks 310/390, 320/391 are preferably initially sewn onto the material plies 31, 32 and the material plies 31, 32 and the middle layer 33 are then joined.

In the variant according to FIG. 3, the first material ply 31, the second material ply 32 and the middle layer 33 are jointly formed from a composite material, wherein the first and second conductor tracks 310, 320 and the first and second connecting tracks 390, 391 are sewn onto the composite material.

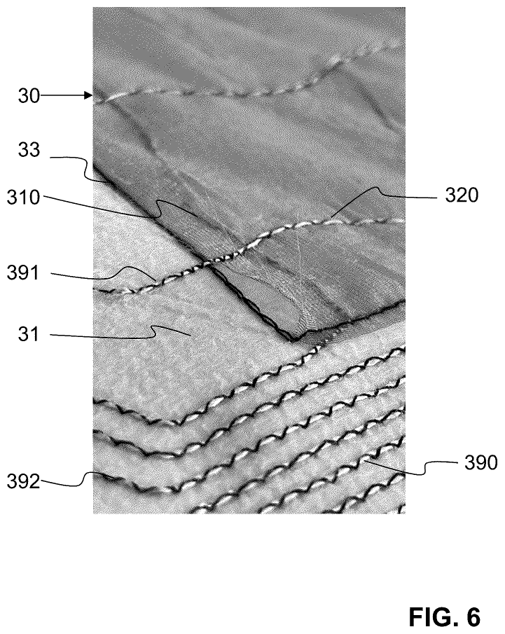

The middle layer 33 and also the first material ply 31, but not a separate second material ply 32, are provided in the embodiment according to FIGS. 5 and 6. The first material ply 31 is preferably a nonwoven material. The middle layer 33 is preferably Carbotex® or another resistance material.

The first material ply 31 projects beyond the middle layer 33 at least in an edge region, preferably on at least two or all of the edge regions. The second conductor tracks 320 are embroidered or sewn onto the middle layer 33. The first conductor tracks 310 are embroidered or sewn onto the first material ply 31. The first and second connecting tracks 390, 391 are secured on the first material ply 31, wherein they are sewn or embroidered onto it. The electronics units, here the flexprints 380, 381, are likewise secured on the first material ply 31. Cables 37 or lines, which lead from the flexprints 380, 381 to the external control device 4, can likewise be secured on the first material ply 31. The flexprints 380, 381 and also the cables 37 or lines can be adhesively bonded, for example, using adhesive strips 6.

The first and second conductor tracks 310, 320 are connected to the respective first and second connecting tracks 390, 391 in one piece in this example. That is to say, they are formed by the same tracks, in particular wires. They are preferably silver-plated copper wires.

Depending on the embodiment, this material composite can be used as such, for example in a mattress or on a bedframe. In other embodiments, it is further surrounded by a protective envelope or it covers at least the middle layer-side surface of the material composite. The envelope preferably consists of the upper ply 35 and/or the lower ply 34.

The embodiment according to FIGS. 5 and 6 is preferably produced as follows: first, the first material webs 310 and the first connecting tracks 390 are sewn or embroidered onto the first material ply 31, here the nonwoven.

The middle layer 33 is subsequently placed onto the first material ply 31. The second conductor tracks 320 are then sewn or embroidered onto the middle layer 33. It is important here that no stitches that could damage the first conductor tracks 310 situated beneath it are made. This is due to programmed embroidery machines which, in the embroidery pattern program, do not make those stitches in each case that would lead to such damage.

The region of the second conductor tracks 320 that projects beyond the middle layer 33, that is to say thus the second connecting tracks 391, are sewn or embroidered onto the first material ply 31 as far as the flexprints 380, 381 in the same working step.

The middle layer 33 is thus likewise secured on the first material ply 31. In other embodiments, it is additionally connected to, in particular sewn on, the first material ply 31 before or after the first conductor tracks are sewn on.

In a next method step, the flexprints 380, 381 are secured on the first material ply 31. This step can also be carried out before the first and second conductor tracks 310, 320 are sewn on or after the first conductor tracks 310 are sewn on, but only after the second conductor tracks 320 are sewn on. After or before the flexprints 380, 381 are secured, the first and second connecting tracks 390, 391 are crimped onto the flexprints 380, 381, the cables 37 are connected to the flexprints 380, 381, if this has not already been done, and guided to the outside.

This embodiment allows industrial production at a minimal cost.

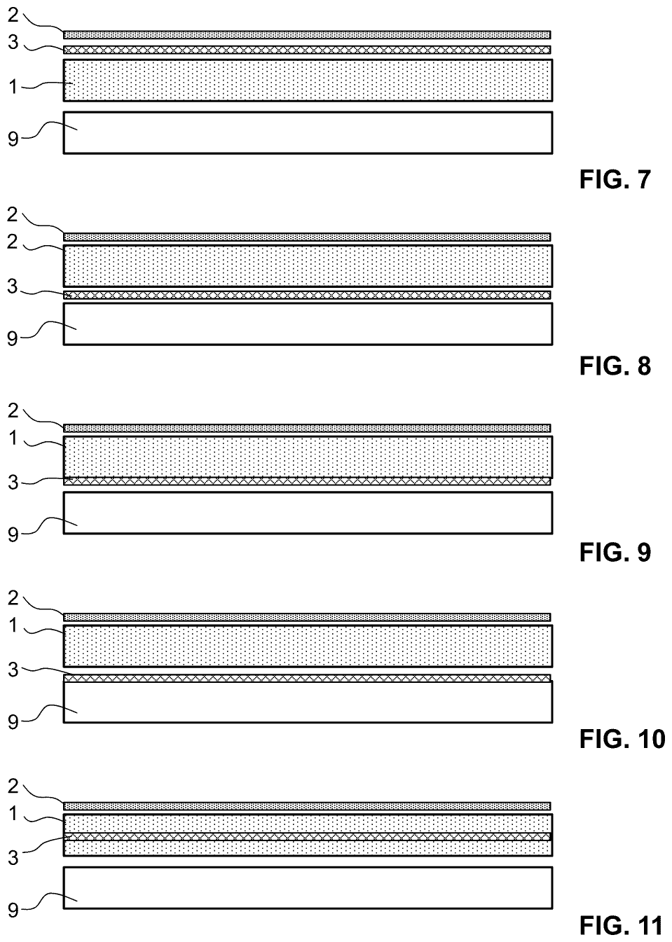

In the described example, the pressure sensor sheet 3 according to the invention is arranged between the bedsheet 2 and the mattress 1. This is once again schematically shown in FIG. 7.

However, the pressure sensor sheet 3 can also be arranged beneath the mattress 1, as a result of which it is arranged between the mattress 1 and a bedframe 9. This is shown in FIG. 8.

Furthermore, it can be integrated into the bottom face of the mattress 1, as can be seen in FIG. 9.

In the variant according to FIG. 10, the pressure sensor sheet 3 is formed jointly with the bedframe 9. It can be, for example, fixed on the bedframe 9 at the manufacturing stage.

In the embodiment according to FIG. 11, the pressure sensor sheet 3 is arranged inside the mattress 1. It can be located in the middle, in the upper region or in the lower region with respect to the thickness of the mattress 1. In the illustrated example, it is arranged approximately in the middle.

Bedframes 9 according to the prior art are very different. For example, spring frames, wire frames, sprung bases, slatted frames and sprung wooden frames are known. Since the first and second conductor tracks 310, 320 and also the first and second connecting tracks 390, 391 are sewn on or in the pressure sensor sheet 3 according to the invention, they can be combined to form any desired patterns, provided that there are enough intersection points 30 as sensors. In particular, the intersection points 30 can be arranged at targeted locations.

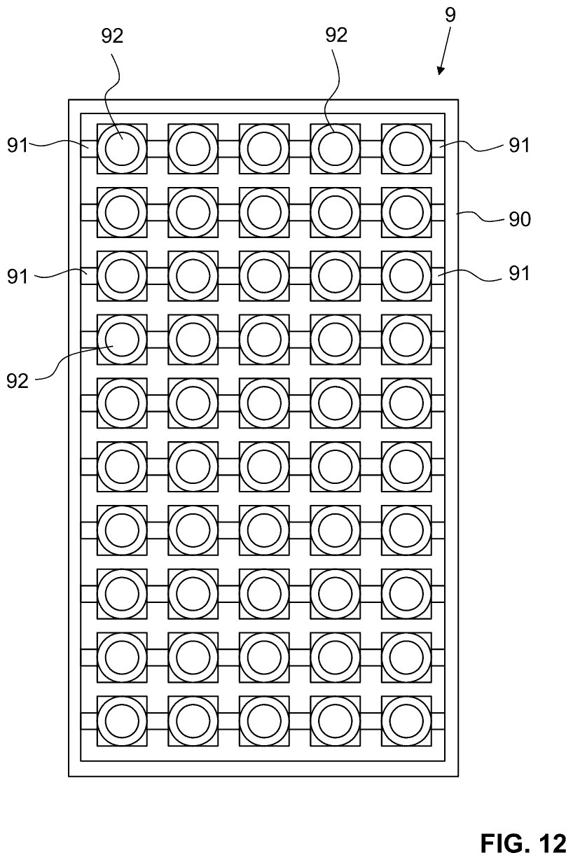

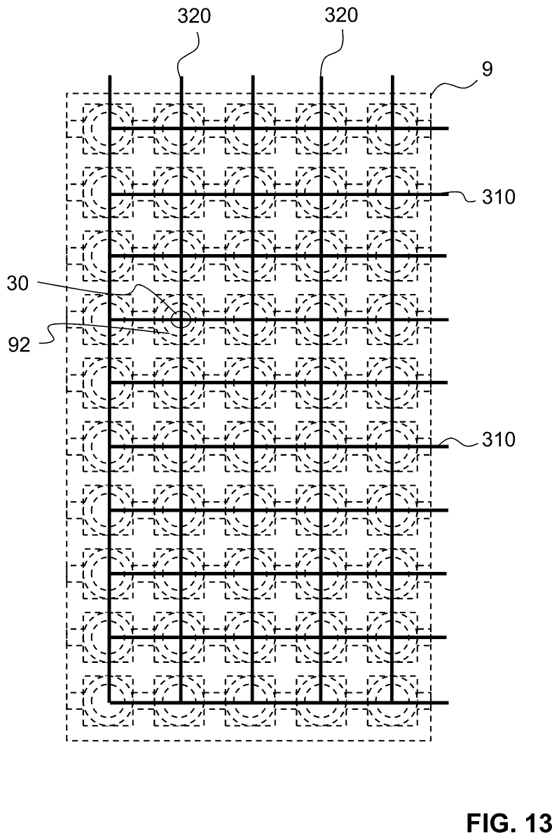

FIG. 12 shows a bedframe 9 having an outer frame 90 and transversely running slats 91. A plurality of plates 92, which adapt in a resilient manner to the loads when the bed is in use, are arranged on the individual slats 91. FIG. 13 shows a pressure sensor sheet 3 according to the invention which is produced in a manner suitable for this bedframe 9, with only the first and second conductor tracks 310, 320 being shown. As can be clearly seen, the spacings between the conductor tracks 310, 320 are selected such that the intersection points 30 each fall on a plate 92.

The pressure sensor sheet 3 according to the invention can be produced in a simple and cost-effective manner and allows flexible configuration both in terms of the choice of material plies 31, 32 and also in terms of the arrangement of the conductor and connecting tracks 310, 320, 390, 391.

LIST OF REFERENCE SIGNS

| | | | |
|---|---|---|---|
| 1 | mattress | 390 | first connecting track |
| 2 | bedsheet | 391 | second connecting track |
| 3 | pressure sensor sheet | 392 | thread |
| 30 | intersection region | 4 | control device |
| 31 | first material ply | 5 | cloud |
| 310 | first conductor track | 6 | adhesive strip |
| 32 | second material ply | 8 | temperature sensor |
| 320 | second conductor track | 80 | sensor line |
| 33 | middle layer | 9 | bedframe |
| 34 | lower ply | 90 | frame |
| 35 | upper ply | 91 | slat |
| 36 | connection | 92 | plate |
| 37 | cable | P | patient |
| 380 | first flexprint | | |
| 381 | second flexprint | | |

1. A flat pressure sensor unit, comprising first conductor tracks and second conductor tracks and comprising a middle layer arranged between the first and the second conductor tracks, wherein the first and second conductor tracks intersect in a manner spaced apart by the middle layer, as a result of which intersection points are formed in order, as sensors, to detect a change in spacing between the first and second conductor tracks, the change in spacing being generated by external application of pressure, wherein the conductor tracks are sewn onto at least one material ply and wherein first and second connecting tracks are provided, which, in a manner leading away from the first and second conductor tracks, extend to at least one electronics unit and end at the at least one electronics unit, and
wherein the first and second connecting tracks are sewn on the at least one material ply.

2. The flat pressure sensor unit as claimed in claim 1, wherein the first and second conductor tracks and also the first and second connecting tracks are secured to the at least one material ply solely by being sewn onto the at least one material ply.

3. The flat pressure sensor unit as claimed in claim 1, wherein extensions of the first and second conductor tracks form the first and second connecting tracks, as a result of which the first and second connecting tracks are formed in one piece with the first and second conductor tracks.

4. The flat pressure sensor unit as claimed in claim 1,
wherein the first conductor tracks form rows of a grid, wherein the rows have ends define two mutually opposite first faces of the grid,
wherein the second conductor tracks form columns of the grid, wherein the columns have ends which define two mutually opposite second faces of the grid,
wherein the first connecting tracks, which lead away from the first conductor tracks, lead along at least one of the two first faces of the grid to at least one of the two second faces of the grid, and
wherein the first and second connecting tracks are connected to the at least one electronics unit on the at least one second face of the grid.

5. The flat pressure sensor unit as claimed in claim 1, wherein the first and second connecting tracks are electrically conductive threads, wires or cables.

6. The flat pressure sensor unit as claimed in claim 5, wherein the surfaces of the first and second conductor tracks and also the first and second connecting tracks do not have any electrical insulation.

7. The flat pressure sensor unit as claimed in claim 1, wherein the flat pressure sensor unit has at least one flexprint, which forms the electronics unit.

8. The flat pressure sensor unit as claimed in claim 1, wherein the first and second connecting tracks are connected to the at least one electronics unit by crimping.

9. The flat pressure sensor unit as claimed in claim 1, wherein the middle layer is a dielectric or is electrically insulating.

10. The flat pressure sensor unit as claimed in claim 1, wherein the first conductor tracks, the first connecting tracks and the second connecting tracks are sewn on a first material ply and the second conductor tracks are sewn onto the middle layer.

11. The flat pressure sensor unit as claimed in claim 1, wherein the first conductor tracks and the first connecting tracks are sewn on a first material ply and the second conductor tracks and the second connecting tracks are sewn on a second material ply, and wherein the middle layer is arranged between the first and the second material ply.

12. The flat pressure sensor unit as claimed in claim 11, wherein the first material ply, the second material ply and the middle layer are self-supporting material webs, which are joined after the first and second conductor tracks and the first and second connecting tracks are sewn on.

13. The flat pressure sensor unit as claimed in claim 1, wherein the first and, if present, the second material ply are a respective or a common textile material ply.

14. A method for producing a flat pressure sensor unit as claimed in claim 1, wherein the first and second conductor tracks are sewn onto at least one material ply in a first specified pattern and wherein the first and second connecting tracks are sewn onto the at least one material ply in a second specified pattern, wherein they lead from the first and second conductor tracks to the at least one electronics unit and wherein the first and second connecting tracks are connected to the at least one electronics unit.

15. The method as claimed in claim 14, wherein the first conductor tracks and the first connecting tracks are sewn onto one material ply, wherein the second conductor tracks are subsequently sewn onto the middle layer and wherein the second connecting tracks are subsequently or simultaneously sewn onto the material ply.

16. The flat pressure sensor unit as claimed in claim 1, wherein the first and, if present, the second material ply are a woven fabric, a warp-knitted fabric, a milled material, a knitted fabric, a mesh, a knitted mat, a nonwoven material or a felt.
